(19) United States
(12) Patent Application Publication
(10) Pub. No.: US 2020/0188405 A1
(43) Pub. Date: Jun. 18, 2020

(54) COMPOSITION FOR TREATING OCULAR DISORDERS SUCH AS MACULAR DEGENERATION, RETINOPATHY AND GLAUCOMA

(71) Applicant: RHEOSTASIS, LLC, Gainesville, FL (US)

(72) Inventor: Shalesh KAUSHAL, Gainesville, FL (US)

(21) Appl. No.: 16/799,956

(22) Filed: Feb. 25, 2020

Related U.S. Application Data
(62) Division of application No. 16/177,571, filed on Nov. 1, 2018.
(60) Provisional application No. 62/582,189, filed on Nov. 6, 2017.

(51) Int. Cl. A61K 31/5377; A23L 33/105; A23L 33/10; A61K 9/08; A23L 33/12; A23L 33/155; A23L 33/16; A23L 33/175; A61P 27/06; A61K 9/00; A61K 45/06; A61K 47/02; A61K 31/407; A61K 31/661; A61K 31/382

(52) U.S. Cl. CPC A61K 31/5377; A61K 9/0053; A23L 33/10; A61K 9/08; A23L 33/12; A23L 33/155; A23L 33/16; A23L 33/175; A61P 27/06; A61K 9/0048; A61K 45/06; A61K 47/02; A61K 31/407; A61K 31/661; A61K 31/382; A23L 33/105

(57) ABSTRACT

A method for treating choroidal neovascularization in a patient includes administrating as eye drops into the eye of the patient having choroidal neovascularization a therapeutically effective amount of an aqueous ophthalmic composition that is formulated for topical administration as eye drops. The aqueous ophthalmic composition consists essentially of Timolol Maleate at a concentration of about 0.1% to 0.5%, Dorzolamide at a concentration of about 0.5% to 2.5%, Prednisolone at a concentration of about 1.0% to 3.0%, Ketorolac Tromethamine at a concentration of about 0.4% to 1.2%, and sterile water at a concentration of at least about 90.0%.

### PRIORITY APPLICATION(S)

This is a divisional application based upon U.S. patent application Ser. No. 16/177,571 filed Nov. 1, 2018, which is based upon U.S. provisional patent application Ser. No. 62/582,189 filed Nov. 6, 2017, the disclosures which are hereby incorporated by reference in their entirety.

### FIELD OF THE INVENTION

The present invention relates to the field of eye health, and more particularly, this invention relates to compositions for treating ocular disorders such as macular degeneration and retinopathy.

### BACKGROUND OF THE INVENTION

Retinal disorders such as wet macular degeneration, diabetic retinopathy, diabetic macular edema, myopic choroidal neovascularization and macular edema following retinal vein occlusion represent some of the more serious retinal orders that if left untreated can lead to partial or even total blindness.

Neovascularization within the eye contributes to visual loss in several ocular diseases, the most common of which are proliferative diabetic retinopathy, neovascular age-related macular degeneration, and retinopathy of prematurity (ROP). Together, these three diseases afflict persons in all stages of life from birth through late adulthood and account for most instances of legal blindness.

Diabetic retinopathy is the leading cause of blindness in adults of working age. In persons with diabetes mellitus, retinal capillary occlusions develop, creating areas of ischemic tissue damage on the retina. Retinal ischemia serves as a stimulus for neovascular proliferations that originate from pre-existing retinal venules at the optic disk or elsewhere in the retina posterior to the equator. Severe visual loss in proliferative diabetic retinopathy (PDR) results from vitreous hemorrhage and tractional retinal detachment. Again, laser treatment (panretinal photocoagulation to ischemic retina) may arrest the progression of neovascular proliferations in this disease but only if delivered in a timely and sufficiently intense manner. Some diabetic patients, either from lack of ophthalmic care or despite adequate laser treatment, go on to sustain severe visual loss secondary to PDR. Vitrectomy surgery can reduce but not eliminate severe visual loss in this disease.

Age-related macular degeneration (AMD) is the leading cause of severe vision loss in persons over 65 years old. In contrast to ROP and PDR, in which neovascularization emanates from the retinal vasculature and extends into the vitreous cavity, AMD is associated with neovascularization originating from the choroidal vasculature and extending into the sub-retinal space. Choroidal neovascularization causes severe vision loss in AMD patients because it occurs in the macula, the area of retina responsible for central vision. The underlying stimuli which lead to choroidal neovascularization are not well understood. Laser ablation of the choroidal neovascularization may stabilize vision in selected patients. However, only 10% to 15% of patients with neo-vascular AMD having lesions are judged to be appropriate for laser photocoagulation according to current criteria.

Retinopathy of prematurity, proliferative diabetic retinopathy, and neo-vascular age-related macular degeneration are but three of the ocular diseases that can produce visual loss secondary to neovascularization. Other ocular diseases include sickle cell retinopathy, retinal vein occlusion associated with macular edema and certain inflammatory diseases of the eye such as glaucoma. Additional treatments beyond laser photocoagulation (e.g., Vertiporfin plus photodynamic laser light therapy) and vitrectomy surgery have been used to improve outcomes in these patients.

Glaucoma, the second leading cause of blindness globally, is an eye disease associated principally with increased intraocular pressure and can be characterized in two basic forms, namely, open-angle glaucoma and angle-closure glaucoma. In the West the most common form of glaucoma is the open-angle variety. In the USA there are approximately 2 million people who suffer from glaucoma annually. It is principally caused by blockage of the anterior chamber's so-called trabecular meshwork which allows excess aqueous humor to drain for the eye to achieve homoeostasis of intraocular pressure (IOP). When the meshwork is partially or totally blocked IOP continues to rise and if left untreated leads to total blindness due to IOP induced optic nerve damage. Early detection of glaucoma is essential for prevention of blindness and many drug, laser and surgical treatments are available to slow the progression of the disease. Drugs are generally used as the first line of defense in treating the IOP symptoms of open-angle glaucoma. These include prostaglandin analogs such as bimatoprost, latanoprost, tafluprost and travoprost. These prostamide analogs mode of action is to increase flow of aqueous humor from the ciliary body and via the uvesclera routes. Beta Blockers have also been employed for the control of IOP and in particular betaxolol and timolol. These drugs reduce the amount of fluid the eye manufactures. Alpha adrenergic agents have also been used for controlling IOP and in particular apraclonidine and brimonidine are effective in improving the removal of fluid from anterior chamber much like the synthetic steroid analogs mentioned earlier. Finally carbonic anhydrase inhibitors have also been used, though rarely, for the control of glaucoma related IOP increases because they also reduce the production of eye fluid. In addition when drug therapy fails to adequately lower IOP, surgical interventions such as trabeculoplasty, canaloplasty, irdotomy and cytophotocoagulation may be employed. Information relating to the disease, causes, treatments and outcomes can be found at www.aao.org/eye-health/diseases/what-is-glaucoma, the website of the American Academy of Ophthalmology, as well as the National Eye Institute and the Glaucoma Research Foundation websites. It is clear that causes of glaucoma are not related to the neovascularization discussed above but rather to the uncontrolled increase of IOP leading to optic nerve damage due to insufficient elimination of fluids from the eye. It is the resulting IOP driven optic nerve damage that leads to partial or total loss of vision.

In recent years, Vascular Endothelial Growth Factor (VEGF) has captured the attention of many investigators involved with ocular neovascularization. The VEGF family of growth factors includes dimeric glycoproteins, which induce endothelial mitogenesis and increase vascular permeability leading to neovascularization. The American Macular Degeneration Foundation has studied and reported extensively on this issue and its website (www.macular.org) has further background information related to wet macular degeneration, its causes and the various treatments used to treat the disease.

Multiple lines of evidence suggest a role for VEGF in ocular neovascular diseases. For example, VEGF levels are increased in the vitreous humor of patients with proliferative diabetic retinopathy compared to the vitreous humor of nondiabetic subjects. VEGF mRNA expression is also increased in a mouse model of oxygen-induced proliferative retinopathy. Human choroidal fibroblasts and retinal pigment epithelial cells normally express low levels of VEGF. On stimulation with phorbol esters, which activate protein kinase C, the choroidal fibroblast VEGF production increases. Surgically excised choroidal neo-vascular membranes from patients with AMD demonstrate immune-histochemical staining for VEGF and VEGF mRNA by in situ hybridization.

These findings later lead to the advent of monoclonal antibody based drugs which specifically lead to the destruction of over-expressed VEGF Vascular Endothelial Growth Factor (VEGF). These drugs have proven effective in the prevention, the onset and in slowing down the progression of ocular neovascularization, however it is important to point out that these VEGF monoclonal antibody treatments do not stop the over-expression of VEGF, they merely remove the over-expressed VEGF from the eye via repeated intra-ocular injections of the commercially available monoclonal antibodies. Such products include, for example, the antibody based VEGF inhibitors ranibizumab, aflibercept, bevacizumab, and pagaptanib. Further details can be found in the two articles: Michael W. Stewart, Clinical and Differential Utility of VEGF Inhibitors in Wet Age-Related Macular Degeneration: Focus on Aflibercept, Clin Ophthalmol., 6: 1175-1186 (2012); Ba et al., Intravitreal Anti-VEGF Injections for Treating Wet Age-Related Macular Degeneration: A Systematic Review and Meta-Analysis, Drug Des Devel Ther., 9: 5397-5405; 2015. Therefore there is a need to develop new drugs or new drug combinations which decrease the over-expression of VEGF by addressing the underlying causes of VEGF expression, the root cause of neovascularization related diseases.

Most of these VEGF antibody therapies rely on regular intraocular injections of the monoclonal antibody preparation directly into the vitreous humor. This advanced type of treatment is a significant economic burden to patients affected by these ocular disease states and requiring these treatments.

These disease states are further complicated by associated inflammatory cytokine expression such as interleukin-6 and as a result, the inflammatory process is manifest via production of vascular exudates. If left unchecked, these exudates produce intraocular pressure (IOP) which, if severe enough, leads to permanent retinal and/or optic nerve damage, leading to partial or total blindness. Therefore it is important to control the expression of VEGF and control IOP in most retinal disease states since neovascularization and its related underlying inflammation can lead to irreversible retinal and optic nerve damage. Further information can be found in Bressler et al., Repeated Intravitreous Ranibizumab Injections for Diabetic Macular Edema and the Risk of Sustained IOP Elevation or Ocular Hypotensive Treatment, JAMA Opthalmol.; 133(5): 589-597 (2015).

Intraocular pressure associated with glaucoma or vascular neogenesis can be controlled by the use of topical ophthalmic preparations of carbonic anhydrase inhibitors (CAI's) either alone or in combination with certain beta blockers. Examples of such products include an ophthalmic topical application of Dorzolamide hydrochloride (a CAI) either alone (e.g., Trusopt®) or in combination with Timolol Maleate (e.g., Cosopt®). Further information on Trusopt and Cosopt can be found in the literature and generally at

www.drugs.com/pro/trusopt.html, having detailed information on the use of Trusopt for the control of IOP, and
www.drugs.com/cosopt.html, having detailed information on the use of Cosopt for the control of IOP.

Timolol Maleate is a topically active non-selective beta blocker that does not have significant intrinsic sympathomimetic, direct myocardial depressant, or local anesthetic activity. The Dorzolamide acts by inhibiting carbonic anhydrase in the ciliary processes of the eye, thus decreasing aqueous humour secretion purportedly by decreasing the formation rate of bicarbonate ions. This results in reduction of both sodium and fluid transport while Timolol Maleate's mechanism of action may be related to the reduction of the formation of aqueous humour in the ciliary body. Information and literature on Timolol Maleate is extensive and general information can be found at different websites, including www.drugs.com/pro/timolol-ophthalmic.html, which includes further detailed information on Timolol (Trusopt) for the control of IOP. Timolol Maleate was also the first β (beta) blocker approved for topical use in treatment of glaucoma in the USA and it is 5-10 times more potent a β blocker than propranolol when used in these ocular applications. Timolol Maleate is also used in combination with brimonidine (e.g.,) Combigan®), an α₂ adrenergic agonist for reduction of ocular hypertension. The literature and general information can be found on different websites, including www.drugs.com/combigan.html, having detailed information on the combination of timolol and brimonidine for the control of IOP. Alpha agonists, through the activation of a G-protein-coupled receptor, inhibit the activity of adenylate cyclase. This reduces cAMP production and hence aqueous humour production by the ciliary body. Peripheral α₂ agonist activity, however, results in vasoconstriction of blood vessels. This vasoconstriction may explain the acute reduction in aqueous humour flow. For further information, see Caprioli et al., The Adenylate Cyclase Receptor Complex and Aqueous Humor Formation, Yale J Biol Med., May-June 1984; 57(3): 283-300.

Ocular inflammation can be treated by the use of corticosteroids as part of ophthalmic preparations such as dexamethasone, prednisolone acetate, triamcinolone acetonide, flurmetholone, betamethazone or loteprednol etabonate (e.g., Lotemax®) or in severe cases of intraocular inflammation, as an implant. Further information can be found in Tempest-Roe et al., Local Therapies for Inflammatory Eye Disease in Translation: Past, Present and Future, BMC Ophthalmol., 13:39 (2013).

Tobramycin, an aminoglycoside, in combination with the steroid dexamethasone (Tobradex®), has been developed for the treatment of ocular infections and attendant inflammation. However, the use of corticosteroid eye drops, especially in long-term use is contraindicated due to reported side effects including hypersensitivity, secondary eye infections due to steroid depression of the ocular surface immune system, the formation of cataracts and increased intraocular pressure (IOP) leading to glaucoma. Consequently, Tobradex® should not be applied for longer than 24 days without further medical evaluation. Information on Tobradex® for control of ocular inflammation is readily available and general information may be found at the website www.drugs.com/pro/tobradex.html.

### SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

The inventor has discovered a combination drug therapy as a composition that in a preferred example is an aqueous ophthalmic composition for treating ocular disorders, such as macular degeneration or retinopathy, and is used alone or in combination with nutraceuticals, to prevent, mitigate, treat and even reverse many of the serious retinal eye diseases indicated above, and more particularly, intraocular neovascularization such that patients do not require intraocular injections of VEGF inhibitors. The composition is also quite effective for the control of glaucoma.

An ophthalmic composition for treating ocular disorders comprises an effective amount of an anti-inflammatory steroid, an NSAID, a beta blocker and a carbonic anhydrase inhibitor, which may be formulated as an aqueous solution used in eye drops or even taken orally, and an antibiotic, which may be topically or orally administered. The anti-inflammatory steroid may comprise one or more of prednisolone sodium phosphate, prednisolone acetate, dexamethasone etabonate, water soluble dexamethasone phosphate and water soluble salts of dexamethasone. The Prednisolone Sodium Phosphate may be at a concentration of about 1.0% to 3.0% in an aqueous solution as an example. The NSAID may comprise one or more of Ketorolac Tromethamine, ibuprofen, and naproxen. The Ketorolac Tromethamine may be at a concentration of about 0.4% to 1.2% in an aqueous solution. The beta-blocker may comprise one or more of Timolol Maleate, Acetazolamide, and Methazaolamide. The Timolol Maleate may be at a concentration of about 0.1% to 0.5% in an aqueous solution. The antibiotic may comprise minocycline or doxycycline. The carbolic anhydrase inhibitor may comprise one or more of Dorzolamide, acetazolamide, dichlorphenamide, methazolamide and brinzolamide. The Dorzolamide may be at a concentration of about 0.5% to 2.5% in an aqueous solution. The composition may be formulated as an aqueous ophthalmic composition containing at least about 90% sterile water.

The composition may be formulated to treat ocular disorders, including macular disorders, retinal neovascular disease states and high intraocular pressure caused by an eye disorder. The composition may be formulated in an oral dosage form. The composition may be formulated to be included within or delivered with an ocular dietary supplement composition or the composition may include one of more of beta carotene, lutein, zeaxanthin in its cis and/or trans forms, astaxanthin, vitamin C, vitamin D, vitamin A, copper, zinc, alpha lipoic acid, N-acetyl carnitine, N-acetylcysteine, and sources of omega-3 fatty acids and esters including but not limited to a marine oil, including fish oil and krill oil. The composition may be formulated as a food ingredient in combination with one or more of kale, broccoli, spinach, brussel sprouts, collard greens, Swiss chard, blueberries, blackberries, raspberries, and pomegranate.

In yet another example, an aqueous ophthalmic composition for treating ocular disorders comprises Timolol Maleate at a concentration of about 0.1% to 0.5%, Dorzolamide at a concentration of about 0.5% to 2.5%, Prednisolone Sodium Phosphate at a concentration of about 1.0% to 3.0%, Ketorolac Tromethamine at a concentration of about 0.4% to 1.2%, and sterile water at a concentration of at least about 90.0%.

In an example, the composition may further comprise valproic acid (a drug better know for its utility as an anti-seizure medication, which the inventor has found and separately reported exhibits profound ocular anti-inflammatory and anti-apoptotic activities) at a concentration of about about 0.5% to 1.5%. A buffering agent may be formulated to obtain a therapeutically acceptable pH of about 7.0 to 7.8 pH. Sodium chloride may be in an amount sufficient to obtain a formulation having a physiologically acceptable osmolality for topical administration as eye drops. A preservative may be in an amount sufficient to obtain a stabilized formulation.

In yet another example, the composition may further comprise an mTOR inhibitor in an effective amount to induce autophagy and reduce druzen formation. An example of such mTOR inhibitor is rapamycin. The composition may be formulated to treat ocular disorders including macular disorders, retinal neovascular disease states and high intraocular pressure caused by an eye disorder. The composition may further include an orally or topically active antibiotic, which in an example, can be taken orally. The composition may be formulated as an adjunct therapy to injectable VEGF antibody inhibitor therapies to significantly reduce the number of annual VEGF inhibitor intraocular injections.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

The inventor is not aware of any single or combined drug therapy that as a composition or formulation, either alone or in combination with nutraceuticals, prevents, mitigates, treats or even reverses many of the serious retinal eye diseases indicated above, and more particularly, intraocular neovascularization. These retinal diseases such as intraocular vascularization require intraocular injections of VEGF inhibitors and do not provide a more holistic approach to disease treatment.

Although tobramycin has been combined with dexamethasone (Tobradex®) and timolol has been separately combined with Dorzolamide (Cosopt®) and widely used for the treatment of ocular hypertension and ocular inflammation, there is no known suggestion in the literature that a combination of all four pharmaceutical products or product groups might be effective in controlling neovascularization or the underlying causes of glaucoma. It would not be technically feasible that one skilled in the art, knowing the biochemistry and chemical pathways of such components, would understand or even have a suggestion that their combination in specific ranges would even be beneficial or effective in controlling neovascularization or glaucoma.

The inventor has surprisingly found that the combination of specific components forming a sterile topical ophthalmic pharmaceutical formulation containing a corticosteroid, a preferably non-selective beta adrenergic receptor blocker or a selective beta adrenergic receptor blocker, a carbonic anhydrase inhibitor and an NSAID surprisingly and unexpectedly prevents, retards, treats and even reverses neovascularization while controlling the underlying processes driving these unwanted angiogenic disease states. Furthermore this same formulation also controls the underlying causes of glaucoma. Therefore, and principally, in many subjects where neovascularization is occurring, there is no longer a need for intraocular injections of VEGF monoclonal antibody therapies since the instant invention as a composition in its sterile topical form can in most cases replace VEGF monoclonal antibody therapies completely or reduce the number of annual injections of VEGF inhibitors by employing the composition as an adjunct therapy to monoclonal antibody VEGF therapy. Since the composition as a pharmaceutical formulation includes a corticosteroid which down regulates ocular surface immunity, it is recommended to co-administer the topical ophthalmic treatment described generally above with an oral antibiotic such as minocycline, a broad spectrum tetracycline antibiotic, or the oral tetracyclic antibiotic doxycycline, or any number of oral antibiotics that achieve similar results. This antibiotic treatment may further be optionally co-administered with oral ocular nutritional supplement therapies.

Therefore, one objective of the invention is to prepare an effective composition that may be a sterile topical ophthalmic treatment such as an aqueous ophthalmic composition useful as eye drops for the prevention, treatment, mitigation and even reversal of such retinal diseases as wet macular degeneration, diabetic retinopathy, diabetic macular edema, myopic choroidal neovascularization and macular edema following retinal vein occlusion, and which may be applied in a single convenient non-intrusive sterile topical dosage form to circumvent the current use of patient unfriendly, expensive, intra-ocular injectable VEGF antibody based inhibitors such as bevacizumab (Avastin®), aflibercept (Eylea®), pagaptanib (Macugen®) and ranibizumab (Lucentis®), and the current use of potent short term corticosteroids, while avoiding the potential side effects of inter-ocular injections.

Another objective is to treat other inflammatory diseases of the eye which result in ocular tissue inflammation using the composition as described.

Yet another objective is the use of the described four-drug composition as described as an orally administered product or a sterile injectable product administered either alone or in combination with antibiotics and/or ocular nutritional supplement therapies.

The composition described herein provides an ocular drug composition that overcomes many of the disadvantages of known drug therapies and methods and treats certain retinal disorders using a unique combination of ingredients as described, either alone or in combination with certain oral antibiotics and/or nutritional ocular therapies. The invention as described herein may be embodied in a topical sterile ophthalmic drug composition and include various methods of preparation. It has utility for the non-invasive treatment of certain ocular disease states and in particular neovascularization. However, it is not intended to be limited to the details described herein because various drug delivery options and/or formulation modifications and pharmaceutical substitutions can be made by those skilled in the art without departing from the spirit of the invention.

Specifically, the composition may be formed as an aqueous ophthalmic composition that is a sterile ocular formulation as an aqueous solution that allows topical application of the combined active pharmaceuticals in a sterile ophthalmic solution to a subject's eye at a concentration that conveniently limits the amount of daily dosing to just 4 drops per eye per day for the prevention, mitigation, treatment and potential reversal of certain ocular disorders. In conjunction with the composition's sterile formulation and oral treatment, minocycline or alternatively doxycycline (or other orally effective antibiotics) are preferably recommended as an adjunct to the topical ophthalmic treatment regimen and may be further enhanced with nutritional supplements related to optimal maintenance of eye health.

Specifically, the composition is an active pharmaceutical formulation approach to ocular treatment, including a mixture of certain key pharmaceuticals as generally described above to form a sterile ophthalmic drop formulation that can be used either alone or preferably in combination with certain oral or sterile topical antibiotic therapies and optionally with oral nutritional supplements known to be associated with eye health, for the prevention, treatment and even reversal of neovascularization processes in the eye.

The aqueous ophthalmic composition as a sterile ocular solution includes a water soluble anti-inflammatory steroid such as Prednisolone Sodium Phosphate, but could be Prednisolone Acetate, a water soluble non-steroidal anti-inflammatory agent (NSAID) such as Ketorolac Tromethamine, a water soluble carbonic anhydrase inhibitor (CAI) such as Dorzolamide, and a water soluble beta blocker such as Timolol Maleate.

The composition in one preferred embodiment that has been successfully, clinically tried includes Timolol Maleate at a concentration of about 0.1% to 0.5% corresponding to about 1 mg/ml to 5 mg/ml. A preferred concentration of Timolol Maleate has been found effective at 1.25 mg/ml or 0.125% on a weight/volume (w/v) basis. Although a preferred range of about 0.1% to 0.5% for Timolol Maleate has been found effective, these ranges can vary depending on application and a patient's susceptibility to a specific component by about 50% above and below the upper and lower ranges of described values and with increments therebetween, such as known 5% increments that may be standardized.

The Dorzolomide has a preferred concentration of about 0.5% to 2.5% corresponding to 5 mg/ml to 25 mg/ml and a preferred 1% concentration corresponding to 10 mg/ml. Again, similar to the Timolol Maleate, the concentration can range above and below these values by about 50% and increments therebetween, for example, 5% increments.

The Prednisolone Sodium Phosphate is at a preferred concentration of about 1.0% to 3.0% corresponding to 10 mg/ml to 30 mg/ml and preferably is at about 20 mg/ml corresponding to a 2.0% solution concentration with variations up to about 50% above and below these values and with increments therebetween as described before. The Letorolac Tromethamine is at a preferred concentration of about 0.4% to 1.2% corresponding to 4 mg/ml to 12 mg/ml and with a preferred 0.8% concentration corresponding to about 8 mg/ml and with variations up to about 50% above and below these values and with increments therebetween as described before. With certain patients, it has been found that Valproic Acid may be added at a concentration of about 0.5% to 1.5% corresponding to 5 mg/ml to 15 mg/ml and with the preferred concentration of about 1%, corresponding to 10 mg/ml. Variations up to 50% above and below these values are possible with increments therebetween as described before. Although Valproic Acid is used typically for treating epilepsy and bipolar disorder, it is advantageous for some patients and aids to help prevent migraine headaches, and thus, with the addition of this Valproic Acid into the ophthalmic composition, it can have salutary benefits for some. Other sodium salts of the Valporic Acid may possibly be used.

A buffering agent is usually included and formulated to obtain a therapeutically acceptable pH of about 7.0 to 7.8 and preferably about 7.4 pH as often used as a guidepost for eye drop compositions. Preservatives may be added in an amount sufficient to obtain a stabilized formulation such as Benzalkonium Chloride, which in one concentration range can be about 0.004% to 0.02% and with ranges therebetween and Boric Acid, which typically may be used as a buffering agent, for example, about 10 mg/ml and varying from 1.0% to 3.0% concentration.

Other examples of preservatives may include chlorobutanol, methyl paraben, sodium perborate, and stabilized thimerosal. Other buffering agents may include disodium-ethylene diamine tetra-acetate (EDTA) and a phosphate-buffered saline. Further details of such buffering agents and preservatives may be found in the article from Epstein et al., Comparative Toxicity of Preservatives on Immortalized Corneal and Conjunctival Epithelia Cells, Journal of Ocular Pharmacology and Therapeutics, Vol. 25, No. 2 (2009). Sodium chloride or other components may be added in an amount sufficient to obtain a formulation having a physiologically acceptable osmolality for topical administration as eye drops.

Those skilled in the art will recognize that the composition as a stable and preferably water-based (aqueous) ophthalmic formulation may contain any water soluble steroid, any water soluble NSAID, any water soluble carbonic anhydrase inhibitor and any water soluble beta blocker that are all selected in effective amounts to function as necessary. Such formulations may also include sterile suspensions of non-water soluble drugs within the same such classes. Such sterile ophthalmic formulations can also be used in conjunction preferably with a broad spectrum oral antibiotic and may further be used either alone or in conjunction with mTOR inhibitors, for example, Rapamycin. Further information about Rapamycin may be found at Li et al., Rapamycin: One Drug, Many Effects, Cell Metab., 19(3): 373-379 (2014). These mTOR inhibitors are known to promote ocular autophagy and may therefore reduce macular druzen formation in addition to the positive effects of the combinatorial active pharmaceutical formulation, which downregulates VEGF activity. Such formulations can be used optionally in conjunction with oral eye healthcare nutraceuticals, including but not limited to: 1) Omega-3 rich fatty acid derivatives derived from fish, krill, algae or plants; 2) cod liver oil rich in Vitamin A; 3) carotenoids such a lutein, zeaxanthin, lycopene, carotene and astaxanthin; 4) turmeric; 5) alpha lipoic acid; 6) oil and/or water soluble antioxidants; and 7) multi-vitamins containing a comprehensive vitamin B complex and the active form of Vitamin D or its related pro-vitamin D form.

Specifically, as noted above, in one exemplary embodiment, the composition as a sterile ophthalmic aqueous solution includes Timolol Maleate preferably at about 0.125%, Dorzolamide preferably at about 1%, Prednisolone Sodium Phosphate (USP) preferably at about 2.0%, and Ketorolac Tromethamine (USP) preferably at about 0.8%, and may be provided to patients in 5 ml and 10 ml amounts for preferred eye drop formulations. This sterile ophthalmic solution may be topically administered to the eye in the preferred droplet form either alone or preferably in conjunction with oral ingestion of an orally effective amount of an antibiotic such as minocycline.

To those skilled in the art, other pharmaceuticals from the same pharmaceutical classes can be substituted for the components described in the preferred formulation and could include at least a water soluble corticosteroid, a water soluble beta blocker, a water soluble NSAID and a water soluble CAI selected in effective amounts to obtain the same or similar results. In an example, it possible to prepare the composition as a formulation by taking the Timolol Maleate and Dorzolamide as commercially available, including powders, formulating and then filtering, and then adding the Prednisolone Sodium Phosphate and Ketorolac Tromethamine as powders and refiltering. The Timolol Maleate and Dorzolamide could be dissolved as powders and run through filters. It possible, however, to start with a commercial formulation of eye drops and fortify with the additional powders for other components, and then sterilize. On a larger scale, it would be advantageous to begin with powders and dissolve them and then filter and place the formulation into a final dosage form. With eye drops, cellulose products, preserving agents, and stabilizers often would be added.

In one specific example, it is possible to prepare the sterile ophthalmic composition as a pharmaceutical preparation using an exemplary preparation method that includes dissolving Ketorolac Tromethamine and Prednisolone Sodium Phosphate in about 2 ml of sterile water for injection. The Ketorolac Tromethamine and Prednisolone solution is then filtered using a 0.22-micron filter into a sterile syringe of appropriate size. An appropriate amount of sterile Timolol Maleate solution of appropriate concentration is drawn into the sterile syringe using aseptic techniques and into the filtered Ketorolac Tromethamine and Prednisolone solution. Next an appropriate aqueous solution of sterile Dorzolamide is added to obtain a final volume of approximately 10 ml and the resulting ophthalmic solution is then dispensed into small sterile single dose droptainers. This solution is suitable for use for up to 3 days at room temperature or 14 days if frozen prior to each use.

Other methods of preparing the composition as an ophthalmic solution will be obvious to those skilled in the art. These methods may include, but are not limited, to the use of the corresponding non-sterile dry pharmaceutically active ingredients which are then dissolved in water for injection, and which may further include the addition of surfactants, components for pH adjustment, sodium chloride to adjust final solution osmolality, and preservatives such as Benzalkonium Chloride to create a stable solution, followed by sterile filtration and then final packaging, most preferably in single dose droptainers as those skilled in the art will appreciate. Single use droptainers are preferred since repeated opening of multi-dose sterile topical preparations often leads to bacterial and fungal contamination which must be avoided in topical eye applications.

As those skilled in the art may appreciate, one or more of the active pharmaceutical ingredients of the ophthalmic composition as the formulation may be delivered in a liposomal preparation for improved bioavailability. Different manufacturing techniques may be used to encapsulate the different components, including use of ultrafiltration and other well-known liposome manufacturing techniques.

As those skilled in the art will recognize, an orally effective combination of each of the active pharmaceutical ingredients from each respective pharmaceutical class as described above may be used instead of the sterile ophthalmic drop formulation to achieve the same or similar results. Such oral formulations may include an orally effective amount of at least a corticosteroid, a NSAID, a beta blocker and a carbonic anhydrase inhibitor which may be used either alone or in combination with an orally effective amount of a broad spectrum antibiotic. In one preferred embodiment as explained above, and without limitation, such orally effective multi-ingredient active pharmaceutical formulation may include an orally effective combination of Prednisolone Acetate, Ketorolac Tromethamine, Timolol Maleate and Dorzolamide Hydrochloride, along with pharmaceutically acceptable excipients to form tablets or capsules or any other acceptable oral formulation presentation. This orally effective formulation is preferably optionally co-administered with an orally effective dose of minocycline, doxycycline or any other broad spectrum antibiotic and optionally co-administered with orally administered eye healthcare nutraceuticals.

The composition as described provides an effective, combinatorial topical drug treatment for the prevention, treatment, mitigation and even reversal of such retinal diseases as wet macular degeneration, diabetic retinopathy, diabetic macular edema, myopic choroidal neovascularization and macular edema following retinal vein occlusion. The composition may circumvent the use of patient unfriendly, expensive, injectable VEGF antibody based inhibitors such as bevacizumab (Avastin), aflibercept (Eylea), pagaptanib (Macugen) and ranibizumab (Lucenis), and avoid the potential side effects and associated risks of inter-ocular injections associated with VEGF inhibitors. The composition may be used to treat other inflammatory diseases of the eye such as glaucoma by addressing each of such disease's respective underlying disease driven mechanisms, which typically result in ocular tissue inflammation or IOP.

As noted before, the standard approach to the treatment of wet AMD has been regular recurring inter-ocular injections of Avastin, a known off-label use and a known VEGF inhibitor since until now there has been no effective alternative treatment for downregulating the progression of this important disease state which ultimately can lead to blindness. To the inventor's knowledge, the literature nowhere describes or reports the use of such a mechanism based multi-ingredient pharmaceutical approach to resolve the underlying cause of ocular diseases, while offering a much more patient acceptable treatment modality, particularly for the prevention, mitigation, treatment and even reversal of eye diseases involving neovascularization.

Those skilled in the art will appreciate that Prednisolone Sodium Phosphate and similar components is an anti-inflammatory steroid medication used to treat certain types of allergies, inflammatory conditions, autoimmune disorders, and cancers, and can be used by mouth, injection into a vein, as a skin cream, and as eye drops. In addition those skilled in the art will appreciate that non-steroidal anti-inflammatory drugs (NSAIDS) downregulate either COX-1, COX-2 or a combination of both pro-inflammatory enzymes. It is well known that COX enzymes are involved in the production of the pro-inflammatory prostaglandins associated with inflammation. Thus, addition of a water soluble NSAID to the composition in combination with an anti-inflammatory steroid like Prednisolone Sodium Phosphate enhances the overall anti-inflammatory effect by downregulating COX mediated inflammatory prostaglandin production.

In certain common retinal diseases, e.g., wet age related macular degeneration (wet AMD), diabetic macular edema (DME) and vein occlusions (either branch or central retinal vein occlusions (BRVO) and CRVO) there can be retinal edema (swelling) within the macula or sub-retinal fluid underneath the macula which, if left untreated, can cause visual cell dysfunction and even vision loss.

Dorzolamide has been found to aid in pumping fluid out of the retina by improving the pump function of the retinal pigment epithelial (RPE) underneath the retina. In addition, it is known that Dorzolamide alone improves retinal and optic nerve blood flow as well as exerting an anti-apoptotic effect on the retina. Certain beta adrenergic receptor antagonists (more commonly known as beta blockers) such as propranolol, when injected into the vitreous humor of human eyes and in animal models have been used for the treatment of certain aspects of some of the common retinal disorders mentioned above. In vitro and in vivo experimentation have been shown to have the ability to downregulate the expression of pro-angiogenic agents like Vascular Endothelial Growth Factor (VEGF) and the cytokine Interleukin-6 (IL-6) which is known to drive expression of VEGF.

Reference should be made for further information to the listed references enumerated below.

Lavine et al., β2-Adrenergic Receptor Antagonism Attenuates CNV Through Inhibition of VEGF and IL-6 Expression, Investigative Ophthalmology and Visual Science, January 2017, Vol. 58, No. 1, 299-308.

Martini et al., Antiangiogenic Effects of β2-Adrenergic Receptor Blockade in a Mouse Model of Oxygen-Induced Retinopathy, J Neurochem., 2011; 119: 1317-1329.

Montero et al., Systemic Beta-Blockers may Reduce the Need for Repeated Intravitreal Injections in Patients with Wet Age-Related Macular Degeneration Treated by Bevacizumab, Retina, 2013; 33(3); 508-512.

Lavine et al., Attenuation of Choroidal Neovascularization by β2-Adrenergic Receptor Antagonism, JAMA Ophthalmol., 2013; 131(3); 376-382.

Ristori et al., Role of the Adrenergic System in a Mouse Model of Oxygen-Induced Retinopathy: Antiangiogenic Effects of β2-Adrenoreceptor Blockade, Invest Ophthalmol Vis Sci., 2011; 52(1); 155-170.

Iaccarino et al., Ischemic Neoangiogenesis Enhanced by β2-Adrenergic Receptor Overexpression: A Novel Role for the Endothelial Adrenergic System, Circ Res., 2005; 97; 1182-1189.

Nourinia et al., Ocular Safety of Intravitreal Propranolol and its Efficacy in Attenuation of Choroidal Neovascularization, Invest Ophthalmol Vis Sci., 2015; 56(13); 8228-8235.

In addition, beta blockers can be retinal neuro-protectants if they can cross the blood-retinal barrier because they reduce the influx of sodium and calcium across voltage sensitive channels. In addition, it is well known that beta blockers improve blood flow. Nevertheless, the reported activities of beta blockers has not lead to the use of beta blockers as a standalone treatment for the control of neo-vascularization in man.

Minocycline was originally identified and developed as a broad spectrum tetracyclic antibiotic. However, as in the case of many drugs, remarkable additional pharmacologically significant properties have been identified by clinicians and scientists. In the instance of minocycline, it is known to be a potent anti-apoptotic agent, scavenges free radicals, chelates calcium, reduces pro-angiogenic activity and most significantly reduces neuro-inflammation by limiting microglial activation. Reference is made to Yao et al., Comparison of Doxycycline and Minocycline in the Inhibition of VEGF-Induced Smooth Muscle Cell Migration, Neurochem Int., February 2007; 50(3): 524-530.

The nutritional therapy that may be used with the composition may include different subsets either singularly or combinations of components. A first subset could include a multivitamin that includes specifically the entire B vitamins, vitamin C, the active form of vitamin D (25-hydroxy 03). The multivitamin may improve overall cellular function and reduce whole body inflammation. A second subset may include alpha lipoic acid, N-acetyl carnitine, and N-acetylcysteine. These three components reduce free radicals that are generated in the body and also improve mitochondrial energy production and improve the intake of omega-3 fatty acids, specifically those found in marine oils such as fish oil and krill oil or cod liver oil. A fourth subset may include the intake of at least one serving a day of dark green vegetables specifically kale, broccoli, spinach, brussel sprouts, collard greens, and Swiss chard. A fifth subset may include the intake of at least one serving a day of dark fruits specifically blueberries, blackberries, raspberries, and pomegranate.

The composition and its use as combinatorial approaches has a two-fold approach, which is accomplished both acutely and locally. Specifically, the drop therapy, along with oral minocycline, is designed to rapidly regulate the inflammatory and proangiogenic activity in the retina. Over time, this systemic combinatorial therapy treats and rectifies the root causes of the diseases that are described above. In one exemplary dosage, the user will apply up to 4 drops of the solution a day for about four days.

Example 1 is a description based on prior knowledge acquired by the inventor during actual clinical use of the preferred composition as described above. Subjects (N=50) underwent a thorough ocular examination and were all found to have varying degrees of choroidal neovascularization and were all deemed professionally to be candidates for standard intraocular injection of VEGF inhibitors to prevent further progression of the disease. All 50 subjects were advised of the sterile ophthalmic multi-ingredient formulation of the invention described herein as well as the alternative standard VEGF inhibitor therapy and were given the choice of either initiating standard VEGF therapy or using the formulation of the invention either as an adjunct therapy to standard VEGF inhibitor therapy or as a substitute for a standard VEGF inhibitor therapy. Of these, 35 subjects without prior VEGF inhibitor therapy elected treatment with the formulation of the invention without VEGF inhibitor therapy. These 35 subjects were treated with 4-6 drops/affected eye/day with the preferred composition of the invention for at least 8 weeks. After the initial 8 weeks of such therapy, their neovascularization progression was evaluated by the inventor who found that 30 of the 35 patients (85%) who had elected the use of the formulation invention instead of the standard VEGF inhibitor therapy had clear evidence of arrested neovascularization. These subjects continued use of the invention formulation for an additional 8 weeks. At the end of the 16 week period, they were re-examined by the inventor and 15 of the 30 subjects were deemed to be in remission from their respective neovascularization (50% of the participating subject subgroup) while the remaining subjects in this sub-group exhibited no further progression of the disease state. The remaining 15 subjects who selected standard VEGF inhibitor therapy, after 8 weeks of such therapy were examined by the inventor and all exhibited partial arrest of progression of their choroidal neovascularization. These subjects (N=15) were offered the added use the formulation invention as an adjunct to their elected standard VEGF inhibitor therapy. Five subjects elected to continue their VEGF inhibitor only therapy for an additional 8 weeks while 10 subjects elected VEGF inhibitor therapy in combination with adjunct therapy with the invention formulation. At the end of the 16 week period, all 15 subjects were again examined by the inventor to evaluate their respective neovascularization progression. The five subjects who elected to continue only their VEGF inhibitor treatments exhibited continued arrested progression of their neovascularization at the end of the 16 week period. The remaining ten subjects who elected the combination of VEGF inhibitor and the formulation invention were also evaluated and all showed either complete arrest or enhanced regression of their respective neovascularization over the subgroup treated only with standard VEGF inhibitors over the same period of time.

The inventor has also worked with patients with confirmed glaucoma and treated them with the composition. In a second example, a number of patients with confirmed glaucoma who were under standard drug therapies, as mentioned previously, were advised of the invention formulation and provided with the option to switch to the invention formulation in lieu of their standard drug therapy. Some of those subjects elected to switch to the invention formulation and were treated for a number of weeks, in an example about 16 weeks at the rate of about 4-6 drops/eye/day. At the end of the initial treatment period, the inventor provided a thorough examination of the affected eyes in all subjects, including but not limited to changes in IOP and field of vision measurements. The subjects who elected to switch to the invention formulation showed dramatic reduction in their respective glaucoma affected eye IOP and importantly exhibited a reduction in the rate of loss of their respective ocular field of vision. When compared to the remaining subjects who elected to continue their standard drug therapy, the invention formulation in the participating subgroup outperformed the standard drug therapy.

It is to be understood that the disclosed embodiments herein are merely exemplary of the invention, which can be embodied in various forms including sterile ophthalmic, oral or injectable formulations. Therefore, specific structural and functional details disclosed herein are not to be interpreted as limiting, but merely as a basis for future development and as a representative basis for teaching one of ordinary skill in the art to variously employ the present invention in virtually any appropriately detailed manner. Further, the terms and phrases used herein are not intended to be limiting, but rather, to provide an understandable description of the invention.

It is to be understood that the terminology used in the description is for the purpose of describing particular embodiments only and is not intended to be limiting. The terms “a” or “an” as used herein are defined as one or more than one. The term “plurality” as used herein is defined as two or more than two. The term “another” as used herein is defined as at least a second or more. The terms “including” and/or “having” as used herein are defined as comprising (i.e., open language). The term “coupled” as used herein is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used in the description, the terms “about” or “approximately” apply to all numeric values, whether or not explicitly indicated. These terms generally refer to a range of numbers that one of skill in the art would consider equivalent to the recited values (i.e., having the same function or result). In many instances, these terms may include numbers that are rounded to the nearest significant figure. While the description herein has several described advantageous features, the composition and method of preparing the same may have other combinations, varying numbers and functions and methods.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A method for treating choroidal neovascularization in a patient, the method comprising administrating as eye drops into the eye of the patient having choroidal neovascularization a therapeutically effective amount of an aqueous ophthalmic composition that is formulated for topical administration as eye drops, the aqueous ophthalmic compostion consisting essentially of:
Timolol Maleate at a concentration of about 0.1% to 0.5%;
Dorzolamide at a concentration of about 0.5% to 2.5%;
Prednisolone at a concentration of about 1.0% to 3.0%;
Ketorolac Tromethamine at a concentration of about 0.4% to 1.2%; and
sterile water at a concentration of at least about 90.0%.

2. The method of claim 1, wherein the aqueous ophthalmic composition includes valproic acid at a concentration of about about 0.5% to 1.5%.

3. The method of claim 1, wherein the aqueous ophthalmic composition includes a buffering agent formulated to obtain a therapeutically acceptable pH of about 7.0 to 7.8 pH.

4. The method of claim 1, wherein the aqueous opthlamic composition includes a salt in an amount sufficient to obtain a formulation having a physiologically acceptable osmolality for topical administration as eye drops.

5. The method of claim 1, wherein the aqueous ophthalmic composition includes a preservative in an amount sufficient to obtain a stabilized formulation.

6. The method of claim 1, wherein the aqueous ophthalmic composition includes an mTOR inhibitor in an effective amount to induce autophagy and reduce druzen formation.

7. The method of claim 1, wherein the aqueous ophthalmic composition is formulated as an adjunct therapy to injectable VEGF antibody inhibitor therapies to significantly reduce the number of annual VEGF inhibitor intraocular injections.

8. The method of claim 1, wherein the Timolol Maleate is at a concentration of about 0.125%.

9. The method of claim 1, wherein the Dorzolamide is at a concentration of about 1.0%.

10. The method of claim 1, wherein the Prednisolone is at a concentration of about 2.0%.

11. The method of claim 1, wherein the Ketorolac Tromethamine is at a concentration of about 0.8%.

12. The method of claim 1, wherein the method includes administering an orally or topically active antibiotic to the patient as an adjunct therapy to the eye drops application of the aqueous ophthalmic composition.

13. A method for treating choroidal neovascularization in a patient, the method comprising administrating for at least eight weeks at least two eye drops and up to six eye drops a day into the eye of the patient having choroidal neovascularization a therapeutically effective amount of an aqueous ophthalmic composition that is formulated for topical administration as eye drops, the aqueous ophthalmic compostion consisting essentially of:
Timolol Maleate at a concentration of about 0.1% to 0.5%;
Dorzolamide at a concentration of about 0.5% to 2.5%;
Prednisolone at a concentration of about 1.0% to 3.0%;
Ketorolac Tromethamine at a concentration of about 0.4% to 1.2%; and
sterile water at a concentration of at least about 90.0%.

14. The method of claim 13, wherein the aqueous ophthalmic composition includes valproic acid at a concentration of about about 0.5% to 1.5%.

15. The method of claim 13, wherein the aqueous ophthalmic composition includes a buffering agent formulated to obtain a therapeutically acceptable pH of about 7.0 to 7.8 pH.

16. The method of claim 13, wherein the aqueous opthlamic composition includes a salt in an amount sufficient to obtain a formulation having a physiologically acceptable osmolality for topical administration as eye drops.

17. The method of claim 13, wherein the aqueous ophthalmic composition includes a preservative in an amount sufficient to obtain a stabilized formulation.

18. The method of claim 13, wherein the aqueous ophthalmic composition includes an mTOR inhibitor in an effective amount to induce autophagy and reduce druzen formation.

19. The method of claim 13, wherein the aqueous ophthalmic composition is formulated as an adjunct therapy to injectable VEGF antibody inhibitor therapies to significantly reduce the number of annual VEGF inhibitor intraocular injections.

20. The method of claim 13, wherein the method includes administering an orally or topically active antibiotic to the patient as an adjunct therapy to the eye drops application of the aqueous ophthalmic composition.
